(19) United States
(12) Patent Application Publication
(10) Pub. No.: US 2015/0276731 A1
(43) Pub. Date: Oct. 1, 2015

(54) Filtration Detection Device and Use Thereof

(71) Applicant: BEIJING COREWAY SCI-TECH COMPANY LTD, Beijing (CN)

(72) Inventor: Marvin Liu, Diamond Bar, CA (US)

(73) Assignee: Marvin Liu, Diamond Bar, CA (US)

(21) Appl. No.: 14/440,347

(22) PCT Filed: Nov. 22, 2013
(86) PCT No.: PCT/CN2013/001433

§ 371 Date: May 1, 2015

(30) Foreign Application Priority Data
Nov. 25, 2012 (CN) 2012206863.6

(51) Int. Cl. G01N 33/543

(52) U.S. Cl. CPC G01N 33/54393; G01N 33/54373

(57) ABSTRACT

A filtration test device and use thereof, comprising a to-be-tested sample container (2), a test phase container (3), a filter (1, 10) and a tester (4); the filter (1, 10) consists of an inlet (5), a filtration layer (6), and an outlet (7); a filtration core in the filtration layer (6) is a solid phase material coupling with the specific conjugate of a to-be-tested object; the to-be-tested sample container (2) and the test phase container (3) respectively communicate with the inlet (5) of the filter (1, 10) via a separate pipe (8, 9). The test device employs the filter (1, 10) as a reaction carrier, improves test customization controllability and test efficiency, significantly reduces reaction time and improves test speed compared with an existing test technique, and can complete the whole reaction at room temperature without a temperature-controlled reaction structure required in an existing test device, thus simplifying instrument design, realizing miniaturization and portability, and having a good application prospect and great significance in improving existing immunoassay techniques.

### TECHNICAL FIELD

The present invention relates to a filtration detection device and use thereof.

### BACKGROUND

Immunological detection technology is an experimental means designed using immunological principles to determine antigens, antibodies, immune cells, as well as chemical ingredients, and the like, and is widely used for samples from human and animal bodies on which disease diagnosis and health assessment may be performed, and for samples utilized in environmental, pharmaceutical, food, and industrial analyses. Commonly used are immunoturbidimetry, solid-phase enzyme immunoassay, chemiluminescence assay, immunofluorescence labeling, flow cytometry, colloidal gold, and the like.

Immunoturbidimetry, also referred to as immunoturbidimetric assay, is to measure light that is transmitted or scattered due to a light refraction or absorption through a complex of a certain size formed by specific binding of an antibody to a soluble antigen in a liquid phase, as base of calculation for an quantitative assay. However, it is low sensitive in detection, not suitable for a microscale assay. Solid-phase enzyme immunoassay is based on immobilization and enzyme-labeling of an antigen or antibody. The antigen or antibody bound to a surface of a solid phase support keeps its immunological activity, and a conjugate of the antigen or antibody with an enzyme maintains both of the immunological activity and the activity of the enzyme. In measurement, a sample to be detected (for detecting an antibody or an antigen therein) and an enzyme-labeled antigen or antibody are allowed to react with the antigen or antibody on the surface of the solid phase support in different steps, which exhibits significant advantages such as high sensitivity, broad linear response range, easy automation, etc. However, the assay has a long reaction time which limits the use thereof. Chemiluminescence immunoassay is a high sensitive microscale and trace analytical way, which has significant advantages such as convenient operation, high sensitivity, broad linear response range, easy automation, etc, and has been widely used in environmental, clinical, pharmaceutical, food and industrial analyses. However, it is a technique likewise based on solid phase isolation and luminescent labeling of an antigen or antibody, and the use thereof is also limited by a long reaction time for assay and a high requirement of detection equipment. Immunofluorescence labeling, flow cytometry, and colloidal gold technology are also commonly used means in assay and have been widely used, but each has corresponding disadvantages.

Currently, there is a trend of developing a high sensitive, quick, downsized, totally quantitative, and automated product for a clinical immunoassay, but none of the existing ones can achieve all of above functions. Thus, the development of a new detection means that is capable of achieving both of the characteristics of being high sensitive, totally quantitative and automated, and the characteristics of rapid detection and equipment miniaturization and portability, will not only permit convenient use and reduced waste, but also can significantly improve efficacy, and thus will have great practical significance in many fields in relation to detection, analysis and isolation.

### DISCLOSURE OF INVENTION

A goal of the present invention is to provide a filtration detection device that is high sensitive, totally quantitative, and rapid in detection.

The present invention provides a filtration detection device that employs a filter as a reaction carrier, and is essentially comprised of a reservoir for sample to be detected, a detection phase reservoir, a filter and a detector; wherein the filter consists of an inlet, a filtration layer and an outlet, and the filtration layer has a filtration core therein that is a solid phase material coupled with a specific binder of an object to be detected; the reservoir for sample to be detected and the detection phase reservoir respectively communicate with the inlet of the filter via a pipe.

Therein, the sample to be detected as described may be particularly any of: a sample from a human or animal body on which a disease diagnosis or health check may be performed, and a sample used for environmental, pharmaceutical, food, and industrial analyses. Detecting the sample from human is a primary thing in a clinical detection, and is used for diagnosis, auxiliary diagnosis, prognosis of various diseases, condition monitoring, etc. Detecting the sample from animal is also a primary thing in a clinical detection, and used for the diagnosis, auxiliary diagnosis, prognosis of various diseases, condition monitoring, and food safety detection, etc., in relation to animals.

The detection phase as described is a solution of a detection material; the detection material is a material formed from a specific binder of an object to be detected, labeled with an indicator; the indicator is a material capable of directly or indirectly producing a color or optical variation.

In general, the object to be detected and the specific binder of the object to be detected are any of the combinations of: an enzyme and an inhibitor, an antigen and an antibody, a ligand and a receptor, or the like.

The indicator is usually an enzyme material, a direct or indirect luminescent material, a fluorescent material, a material with its own color, or the like. Generally used enzyme materials comprise horseradish peroxidase (HRP), alkaline phosphatase (ALP), glucose oxidase, β-galactosidase lysozyme, and malate dehydrogenase, and the like. The direct or indirect luminescent material generally used comprises luminol and derivatives thereof, lucigenin, lophine, peroxidated oxalic acid esters, acridine esters, and the like. The fluorescent material comprises fluorescein isothiocyanate (FITC), or rhodamine (RB200), or the like, a material with its own color such as colloidal golds, and the like.

In the device as described, the detector may be an instrument that enables a quantitative detection and a recordation of the color or light quantity variation, and may be particularly any of: an enzyme-linked immunosorbent assay (ELISA) detector, a chemiluminescence detector, a fluorescence detector and a spectrophotometer.

In the device as described, the solid phase material serving as a filtration core may be a particulate material or a mesh-like material. The solid phase particulate or mesh-like material may be various solid materials that are able to couple with an antigen or an antibody while not leading to a significant variation in the immunological binding properties of the antigen or antibody, such as, as generally used, gel particles, latex particles, magnetic particles, and the like; the gel particles comprise dextrane gel-based gel filtration fillers and agarose gel-based gel filtration fillers, including generally used cyanogen bromide-activated agarose gel medium, NHS-activated agarose gel medium, and the like.

In an embodiment of the present invention, the reservoir for sample to be detected and the detection phase reservoir are particularly 5 mL centrifuge tubes; and both of the pipe linking the reservoir for sample to be detected to the filter, and the pipe linking the detection phase reservoir to the filter are particularly silicone tubes.

In the device as described, the filtration core may have a volume of 2 mm³ to 1 cm³, for example, 3 mm³ to 0.3 cm³, or further 5 mm³ to 0.1 cm³, or even further 70-100 mm³. In an embodiment of the present invention, the filtration core has a volume of particularly 70 mm³; in another embodiment of the present invention, the filtration core has a volume of particularly 100 mm³.

In the device as described, the filtration core may be shaped having length greater than width, with a ratio of length to width of 2-100, for example, 2-50, or further 2-30, and may be cyclindrical, conical, cubic, cuboid, and a combination thereof, and the like. Of course, the filtration core also may be shaped having width greater than length, with a ratio of width to length of 1.1-10, for example, 1.1-5, or further 1.1-3, and may be cylindrical, conical, cubic, cuboid, or a combination thereof, or the like.

In an embodiment of the present invention, the filtration core is a long cylinder, with a ratio of length to width (a ratio of height to bottom diameter of the cylinder) of 10:3; in another embodiment of the present invention, the filtration core is a short cylinder, with a ratio of width to length (a ratio of bottom diameter to height of the cylinder) of 10:5.

In the device as described, the pipe communicating between the reservoir for sample to be detected and the filter, and the pipe communicating between the detection phase reservoir and the filter each has a pump (e.g., a peristaltic pump) fixed thereon; the pump (e.g., a peristaltic pump) is used to force liquid transport from the reservoir (the reservoir for sample to be detected or the detection phase reservoir) to the filter, and to control the flow rate during filtration.

In the embodiments of the present invention, a peristaltic pump is used to force liquid transport, and the peristaltic pump is a commercially available from LongerPump Co., Ltd., Baoding, China, under Catalog No. BQ50-1J.

Another goal of the present invention is to provide a method for determining the amount of an object to be detected in a sample to be detected using the filtration detection device described.

The present invention provides a method for determining the content of an object to be detected in a sample to be detected using the filtration detection device described, which may be described as (A) or (B) below:

(A) comprising steps of:

1) placing solution A into the reservoir for sample to be detected; placing solution B into the detection phase reservoir (i.e., a detection phase); the solution A being a solution of a sample to be detected containing the object to be detected; the solution B being a solution of a detection material, the detection material being a material formed from a specific binder of an object to be detected, labeled with an indicator; the indicator being a substance capable of directly or indirectly resulting in a color or light quantity variation;

2) flowing the solution A into the filter through a pipe, so that the object to be detected is specifically captured by the filtration core;

3) flowing the solution B into the filter through a pipe, so that the detection material is specifically bound with the object to be detected that is specifically captured by the filtration core, to form complex 1, a complex of the filtration core, the object to be detected, and the detection material;

4) detecting, as any of following (a)-(c):

(a) detecting the filtration core with the captured object to be detected and detection material directly using a detector, to calculate the content of the object to be detected through a color or light quantity variation produced by the indicator;

(b) eluting the filter with an eluent, collecting the eluent containing the detection material, detecting the eluent with the detector for a color or light quantity variation of the indicator, to thereby calculate the content of the object to be detected;

(c) detecting the solution B flowing out of the filter (not bound) directly using a detector for a color or light quantity variation of the indicator, to thereby calculate the content of the object to be detected;

(B) comprising steps of:

1) placing solution A into the reservoir for sample to be detected; placing solution B1 into the detection phase reservoir; the solution A being a solution of a sample to be detected containing the object to be detected; the solution B1 being a solution of a specific binder of the object to be detected, not labeled with an indicator;

2) flowing the solution A into the filter through a pipe, so that the object to be detected is specifically captured by the filtration core;

3) flowing the solution B1 into the filter through a pipe, so that the specific binder of the object to be detected not labeled with the indicator is specifically bound with the object to be detected specifically captured by the filtration core, to form complex 2, a complex of the filtration core, the object to be detected, and the unlabeled specific binder of the object to be detected;

4) placing solution B2 into the detection phase reservoir; the solution B2 being a solution of a material labeled with an indicator, the material labeled with the indicator being capable of binding with the specific binder of the object to be detected in the solution B1; the indicator being a substance capable of directly or indirectly resulting in a color or light quantity variation;

5) flowing the solution B2 into the filter through a pipe, so that the material labeled with the indicator is bound with the unlabeled specific binder of the object to be detected in the complex 2, to form complex 3, a complex of the filtration core, the object to be detected, the unlabeled specific binder of the object to be detected, and the material labeled with the indicator;

6) detecting, as any of following (a)-(c):

(a) detecting the filtration core with the captured object to be detected, the unlabeled specific binder of the object to be detected and the material labeled with the indicator directly using a detector, to calculate the content of the object to be detected through a color or light quantity variation produced by the indicator;

(b) eluting the filter with an eluent, collecting the eluent containing the material labeled with the indicator, detecting the eluent with the detector for a color or light quantity variation of the indicator, to thereby calculate the content of the object to be detected;

(c) detecting the solution B2 flowing out of the filter directly using a detector for a color or light quantity variation of the indicator, to thereby calculate the content of the object to be detected.

In above method, each of the solutions is flowed into the filter, at a flow rate of 0.05-1.0 mL/min; for example, 0.1-0.3 mL/min, during filtration.

The method may further comprise, after step 2) or 3), a step of cleaning the filter with a cleaning liquid.

Therein, (A) is a generally used two-step process, that is, injecting a solution of a sample to be detected containing the object to be detected into the filter for filtration, and cleaning; followed by injecting a detection material solution that is capable of specifically binding to the object to be detected and of directly or indirectly resulting in a color or light quantity variation into the filter for filtration, and directly detecting. (B) is a three-step process, and may be a more-step process, that is, injecting a solution of a sample to be detected containing the object to be detected into the filter for filtration, and cleaning; then filtering a solution of a specific binder of the object to be detected unlabeled with an indicator (this step is performed at least once); followed by injecting a solution of a material that is capable of binding with the unlabeled specific binder of the object to be detected and of directly or indirectly resulting in a color or light quantity variation into the filter for filtration, and detecting.

In the methods described, the sample to be detected may be particularly any of: a sample from a human or animal body on which a disease diagnosis or health check may be performed, and a sample used for environmental, pharmaceutical, food, and industrial analyses. Detecting the sample from human is a primary thing in a clinical detection, and is used for diagnosis, auxiliary diagnosis, prognosis of various diseases, condition monitoring, etc. Detecting the sample from animal is also a primary thing in a clinical detection, and used for the diagnosis, auxiliary diagnosis, prognosis of various diseases, condition monitoring, and food safety detection, etc., in relation to animals.

In general, the object to be detected and the specific binder of the object to be detected are any of the combinations of: an enzyme and an inhibitor, an antigen and an antibody, a ligand and a receptor, and the like. The indicator generally is an enzyme material, a direct or indirect luminescent material, a fluorescent material, a material with its own color, or the like. Generally used enzyme materials comprise horseradish peroxidase (HRP), alkaline phosphatase (ALP), glucose oxidase, β-galactosidase lysozyme, and malate dehydrogenase, and the like. The direct or indirect luminescent material generally used comprises luminol and derivatives thereof, lucigenin, lophine, peroxidated oxalic acid esters, acridine esters, and the like. The fluorescent material comprises fluorescein isothiocyanate (FITC), or rhodamine (RB200), or the like, a material with its own color such as colloidal golds, or the like.

A still another goal of the present invention is to provide a filter.

The present invention provides a filter that may be used for a quantitative immunoassay, wherein the filter consists of an inlet, a filtration layer and an outlet, and the filtration layer has a filtration core therein that is a solid phase material coupled with a specific binder of an object to be detected.

As the filtration core, the solid phase material may be a particulate matter or a mesh-like material.

In practical use, the solid phase particulate or mesh-like material may be various solid materials that are able to couple with antigens or antibodies and do not lead to a substantial variation in the immunological binding properties of the antigens or antibodies, such as, as generally used, gel particles, latex particles, magnetic particles, and the like; the gel particles comprise dextrane gel-based gel filtration fillers and agarose gel-based gel filtration fillers, including generally used cyanogen bromide-activated agarose gel medium, NHS-activated agarose gel medium, and the like.

In the filter, the filtration core may have a volume of 2 mm³ to 1 cm³, for example, 3 mm³ to 0.3 cm³, or further 5 mm³ to 0.1 cm³.

In the filter, the filtration core may be shaped having length greater than width, with a ratio of length to width of 2-100, for example, 2-50, or further 2-30, and may be cylindrical, conical, cubic, cuboid, or a combination thereof, or the like. Of course, the filtration core also may be shaped having width greater than length, with a ratio of width to length of 1.1-10, [symbol] 1.1-5, or further 1.1-3, and may be cylindrical, conical, cubic, cuboid, or a combination thereof, or the like.

In an embodiment of the present invention, the filtration core is a long cylinder, with a ratio of length to width (a ratio of height to bottom diameter of the cylinder) of 10:3; in another embodiment of the present invention, the filtration core is a short cylinder, with a ratio of width to length (a ratio of bottom diameter to height of the cylinder) of 10:5.

A still another goal of the present invention is to provide a method for detecting the content of an object to be detected in a sample to be detected using the filter described.

The present invention provides a method for detecting the content of an object to be detected in a sample to be detected using the filter described, which may be described as (C) or (D) below:

(C) comprising steps of:

1) placing solution A into the reservoir for sample to be detected; placing solution B into the detection phase reservoir; the solution A being a solution of a sample to be detected containing the object to be detected; the solution B being a solution of the detection material, the detection material being a material formed from a specific binder of an object to be detected, labeled with an indicator; the indicator being a substance capable of directly or indirectly resulting in a color or light quantity variation;

2) flowing the solution A into the filter through a pipe, so that the object to be detected is specifically captured by the filtration core;

3) flowing the solution B into the filter through a pipe, so that the detection material is specifically bound with the object to be detected that is specifically captured by the filtration core, to form complex 1, a complex of the filtration core, the object to be detected, and the detection material;

4) detecting, as any of following (a)-(c):

(a) detecting the filtration core with the captured object to be detected and detection material directly using a detector, to calculate the content of the object to be detected through a color or light quantity variation produced by the indicator;

(b) eluting the filter with an eluent, collecting the eluent containing the detection material, and detecting a color or light quantity variation of the indicator in the eluent using a detector, to thereby calculate the content of the object to be detected;

(c) detecting the solution B flowing out of the filter directly using a detector for a color or light quantity variation of the indicator, to thereby calculate the content of the object to be detected;

(D) comprising steps of:

1) placing solution A into the reservoir for sample to be detected; placing solution B1 into the detection phase reservoir; the solution A being a solution of a sample to be detected containing the object to be detected; the solution B1 being a solution of a specific binder of the object to be detected, not labeled with an indicator;

2) flowing the solution A into the filter through a pipe, so that the object to be detected is specifically captured by the filtration core;

3) flowing the solution B1 into the filter through a pipe, so that the unlabeled specific binder of the object to be detected is specifically bound with the object to be detected specifically captured by the filtration core, to form complex 2, a complex of the filtration core, the object to be detected, and the unlabeled specific binder of the object to be detected;

4) placing solution B2 into the detection phase reservoir; the solution B2 being a solution of a material labeled with an indicator, the material labeled with the indicator being capable of binding with the specific binder of the object to be detected in the solution B1; the indicator being a substance capable of directly or indirectly resulting in a color or light quantity variation;

5) flowing the solution B2 into the filter through a pipe, so that the material labeled with the indicator is bound with the unlabeled specific binder of the object to be detected in the complex 2, to form complex 3, a complex of the filtration core, the object to be detected, the unlabeled specific binder of the object to be detected, and the material labeled with the indicator;

6) detecting, as any of following (a)-(c):

(a) detecting the filtration core with the captured object to be detected, the unlabeled specific binder of the object to be detected and the material labeled with the indicator directly using a detector, to calculate the content of the object to be detected through a color or light quantity variation produced by the indicator;

(b) eluting the filter with an eluent, collecting the eluent containing the material labeled with the indicator, and detecting a color or light quantity variation of the indicator in the eluent using a detector, to thereby calculate the content of the object to be detected;

(c) detecting the solution B2 flowing out of the filter directly using a detector for a color or light quantity variation of the indicator, to thereby calculate the content of the object to be detected.

In above method, the detector may be an instrument that enables the quantitative detection and recordation of the color or light quantity variation, and particularly may be any of: an ELISA detector, a chemiluminescence detector, a fluorescence detector, and a spectrophotometer.

In above method, each of the solutions is injected into the filter, at a flow rate of 0.05-1.0 mL/min; for example, 0.1-0.3 mL/min, during filtration.

The method may further comprise, after step 1) and/or step 2), a step of cleaning the filter with a cleaning liquid.

Therein, (C) is a two-step process as generally used, that is, injecting a solution of a sample to be detected containing the object to be detected into the filter for filtration, and cleaning; followed by injecting a detection material solution that is capable of specifically binding to the object to be detected and of directly or indirectly resulting in a color or light quantity variation into the filter for filtration, and directly detecting. (D) is a three-step process or a more-step process, that is, injecting a solution of a sample to be detected containing the object to be detected into the filter for filtration, and cleaning; then filtering a solution of a specific binder of the object to be detected unlabeled with an indicator (this step is performed at least once); followed by injecting a solution of a material that is capable of binding with the unlabeled specific binder of the object to be detected and of directly or indirectly resulting in a color or light quantity variation into the filter for filtration, and detecting.

In the methods described, the sample to be detected may be particularly any of: a sample from a human or animal body on which a disease diagnosis or health check may be performed, and a sample used for environmental, pharmaceutical, food, and industrial analyses. Detecting the sample from human is a primary thing of a clinical detection, and is used for diagnosis, auxiliary diagnosis, prognosis of various diseases, condition monitoring, etc. Detecting the sample from animal is also a primary thing in a clinical detection, and used for the diagnosis, auxiliary diagnosis, prognosis of various diseases, condition monitoring, and food safety detection, etc., in relation to animals.

In general, the object to be detected and the specific binder of the object to be detected are any of the combinations of: an enzyme and an inhibitor, an antigen and an antibody, a ligand and a receptor, and the like. The indicator generally is an enzyme material, a direct or indirect luminescent material, a fluorescent material, a material with its own color, or the like. Generally used enzyme materials comprise horseradish peroxidase (HRP), alkaline phosphatase (ALP), glucose oxidase, β-galactosidase lysozyme, and malate dehydrogenase, and the like. The direct or indirect luminescent material generally used comprises luminol and derivatives thereof, lucigenin, lophine, peroxidated oxalic acid esters, acridine esters, and the like. The fluorescent material comprises fluorescein isothiocyanate (FITC), or rhodamine (RB200), or the like, a material with its own color such as colloidal golds, or the like.

Use of the filtration detection device or the filter provided by the present invention as described above in the manufacture of a product for a quantitative immunoassay is also intended to be within the protection scope of the present invention.

In an embodiment of the present invention, each of above objects to be detected is particularly human fibrinogen.

In the present invention, each of above samples to be detected is particularly a human fibrinogen solution or in vitro human plasma (in vitro plasma from healthy human).

In the present invention, each of above specific binders of the objects to be detected is particularly a polyclonal antibody against human fibrinogen or a monoclonal antibody against human fibrinogen.

In the present invention, each of above detection materials (corresponding to methods (A) and (C)) is particularly a horseradish peroxidase labeled monoclonal antibody against human fibrinogen or a colloidal gold labeled monoclonal antibody against human fibrinogen. Each of above unlabeled specific binders of the objects to be detected (corresponding to methods (B) and (D)) is particularly a monoclonal antibody against human fibrinogen, and each of the materials labeled with the indicator (corresponding to methods (B) and (D)) is particularly an alkaline phosphatase labeled secondary antibody or a horseradish peroxidase labeled secondary antibody.

In the present invention, each of above detectors is particularly a chemiluminescence detector, an ELISA detector, or a spectrophotometer.

In the present invention, each of above solid phase materials is particularly NHS-activated agarose gel particles; correspondingly, each of above filtration cores is particularly NHS-activated agarose gel particles labeled with a polyclonal antibody against human fibrinogen. Further, during the preparation of the filtration core, the ratio between the polyclonal antibody against human fibrinogen and the NHS-activated agarose gel particles may be 0.1-10 mg: 1 mL (for example, 2.5 mg: 1 mL). The NHS-activated agarose gel particles are particularly commercially available from Beijing Weishi Bohui Chromatography Technology Co., Ltd., China, under CS-A30-01 NHS-activated Sepharose FF.

In the present invention, each of above filters is particularly made following a method comprising steps of:

(a) making a housing of the filter, the material of which may be a plastic (e.g., a hard plastic);

(b) transferring NHS-activated agarose gel particles labeled with a polyclonal antibody against human fibrinogen as a filtration core into the housing of the filter in step (a), to obtain the filter of the present invention.

### DESCRIPTION OF DRAWINGS

FIG. 1 is a schematic diagram of the basic structure of a filtration detection device having a long-cylindrical filter.

FIG. 2 is a schematic diagram of the basic structure of a filtration detection device having a short-cylindrical filter.

### BEST MODE FOR IMPLEMENTING THE INVENTION

Following examples are only intended to facilitate a better understanding of the present invention, but not for limiting the present invention. All of the experimental methods used in following examples are conventional methods, unless being otherwise specifically stated. All of the experimental materials used in following examples are commercially available from conventional biochemical manufacturers, unless being otherwise specifically stated. All of the quantitative assays in following examples are repeated three times, and the results were averaged.

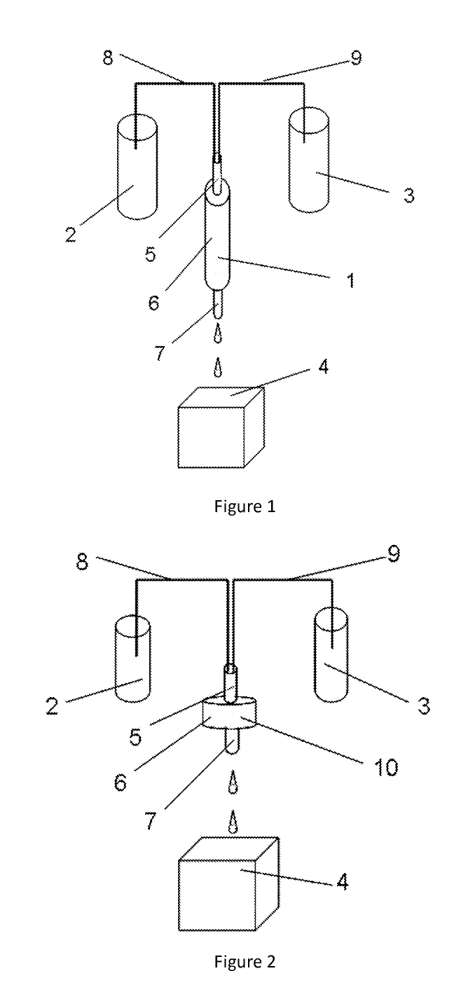

As shown in FIG. 1 and FIG. 2, the filtration detection device provided in the present invention is essentially comprised of a reservoir for sample to be detected 2, a detection phase reservoir 3, a detector 4, and a long-cylindrical filter 1 or a short-cylindrical filter 10. The long-cylindrical filter 1 or short-cylindrical filter 10 each particularly consists of an inlet 5, a filtration layer 6 and an outlet 7, wherein the filtration layer 6 has therein a filtration core which is a labeled solid phase material coupled with a specific binder (e.g., an antigen or antibody) of the object to be detected. The reservoir for sample to be detected 2 and the detection phase reservoir 3 respectively communicate with the inlet 5 of the long-cylindrical filter 1 or the short-cylindrical filter 10 via a pipe 8 and a pipe 9.

In the device as described, the pipe 8 communicating between the reservoir for sample to be detected 2 and filter (the long-cylindrical filter 1 or the short-cylindrical filter 10), and the pipe 9 communicating between the detection phase reservoir 3 and the filter (the long-cylindrical filter 1 or the short-cylindrical filter 10) further have a pump fixed thereon, respectively; the pump is used to force liquid transport from the reservoir (the reservoir for sample to be detected 2 or the detection phase reservoir 3) to the filter, and to control the flow rate during filtration.

The long-cylindrical filter 1 or the short-cylindrical filter 10 is a reaction carrier for detecting reaction, and the main reaction process is performed on the long-cylindrical filter 1 or the short-cylindrical filter 10. As a filtration core, the solid phase material may be a deposit of a solid phase particulate matter that form gaps between particles as filtering pores, and also may be a mesh-like solid phase matter.

The reservoir for sample to be detected 2 is used to hold a sample from a human or animal body on which a disease diagnosis or health check may be performed, and a sample solution used for environmental, pharmaceutical, food, and industrial analyses (containing an object to be detected).

The detection phase reservoir 3, is used to hold a solution of a detection material; the detection material being a material formed from a specific binder of an object to be detected, labeled with an indicator; the indicator being a substance capable of directly or indirectly resulting in a color or light quantity variation.

The detector 4 is an instrument that enables the quantitative detection and recordation of the color or light quantity variation, including, as generally used, an ELISA detector, a chemiluminescence detector, a fluorescence detector, and a spectrophotometer, and the like.

### Example 1

### Preparation of Filtration Detection Device

I. Preparation of Filter

1. Experimental Materials

A self made long cylinder microfilter housing (with a height of 15 mm, and a diameter of 5 mm), NHS-activated agarose gel particles (CS-A30-01 NHS-activated Sepharose FF, Beijing Weishi Bohui Chromatography Technology Co., Ltd., China), an anti-human fibrinogen polyclonal antibody (Commercially available from Yili Gaoke Bioengineering Institute, Beijing, China, Art. No. BR0812), sodium bicarbonate, hydrochloric acid and ethanolamine.

2. Experimental Method

5 mg of the anti-human fibrinogen polyclonal antibody was taken, and added to 2 ml of an aqueous solution of sodium bicarbonate at a concentration of 0.2M (pH 8.3) to dissolve. 1 ml of NHS-activated agarose gel particles were taken, and washed with 20 ml of 1 mM hydrochloric acid in three installments with sucking filtration, with removing all of the hydrochloric acid solution. The solution of the anti-human fibrinogen polyclonal antibody was mixed with the treated NHS-activated agarose gel particles in a volume ratio of 1:1 at 4° C., and allowed for reaction with shaking for 4 hours. After the reaction, the NHS-activated agarose gel particles were washed with pure water, and then added to a solution containing 10 mM ethanolamine and 0.2M sodium carbonate, pH 8.0, shaken at room temperature for 4 hours to block uncoupled groups, and washed with 50 mM phosphate buffer to clean, and the resulting NHS-activated agarose gel particles were made into a filtration core, loaded into the self made long cylinder microfilter housing, which was closed for use. The resulting filtration core is a long cylinder (with a ratio of length to width of 10:3, i.e., a ratio of height to bottom diameter of 10:3 of the cylinder), having a volume of 70 mm³.

II. Preparation of Filtration Detection Device

As shown in FIG. 1. Two 5 ml centrifuge tubes were taken, one used as the reservoir for sample to be detected, and the other used as detection phase reservoir, These two reservoirs were respectively communicated with the filter made in step I at inlet via silicone tubes, while a peristaltic pump (Commercially available from LongerPump Co., Ltd., Baoding, China, under Catalog No. BQ50-1J) was fixed on each of the two silicone tubes, to force liquid transport from the reservoirs to the filter, and to control the flow rate during filtration. The communicated device, together with a detector, formed the filtration detection device provided by the present invention. Therein, the detector was an instrument that enables the quantitative detection and recordation of the color or light quantity variation, including, as generally used, an ELISA detector, a chemiluminescence detector, a fluorescence detector, and a spectrophotometer, and the like.

### Example 2

### Comparison on Detection Performance Between the Invention and the Existing Chemiluminescence Detection Technology

I. Experimental Materials

The anti-human fibrinogen polyclonal antibody filter prepared in Example 1, horseradish peroxidase (HRP, commercially available from Nanjing Dulai Biotech Co., China, Art. No. RZ-3), anti-human fibrinogen monoclonal antibody (Commercially available from Yili Gaoke Bioengineering Institute, Beijing, China, Art. No. BR0616), an anti-human fibrinogen polyclonal antibody (Commercially available from Yili Gaoke Bioengineering Institute, Beijing, China, Art. No. BR0812), glutaraldehyde, magnetic particles (MP-COOH-20020, Yingnuo Biotechnology Co., Ltd., Zhengzhou, China), luminol, p-lodophenol, carbamide peroxide, a chemiluminescence detector (Promega, Glomax MultiJR Detection System), human fibrinogen (Commercially available from Sigma-Aldrich, under Product Catalog No. F3879-1 G).

II. Experimental Method

Formulation of a human fibrinogen solution: a known concentration of human fibrinogen solution was taken, and diluted with a PBS solution to formulate a solution of 1 μg/ml human fibrinogen.

Labeling of magnetic particles: in a conventional labeling way, magnetic particles were labeled with an anti-human fibrinogen polyclonal antibody, with a ratio (w/w) of the amounts of the anti-human fibrinogen polyclonal antibody and the magnetic particles of 1:10.

Formulation of a working solution of chemiluminescent substrate: the working solution of chemiluminescent substrate was formulated with luminol, p-lodophenol, carbamide peroxide and the like. The working solution of chemiluminescent substrate has a solvent of water, and solutes and concentrations thereof as follows: 0.36 mM luminol, 0.35 mM p-lodophenol, 4.5 mM carbamide peroxide. Each of above concentrations is a final concentration of corresponding component in the solution.

Preparation of horseradish peroxidase labeled anti-human fibrinogen monoclonal antibody: in Glutaraldehyde Two-Step process, 25 mg of HRP was weighted, dissolved in a 1.25% glutaraldehyde solution, and left to stand at room temperature overnight. After the reaction, the enzyme solution was passed through Sephadex G-25 chromatography column, eluted with normal saline. 12.5 mg of antibody to be labeled was diluted with normal saline to 5 ml, and added to the enzyme solution dropwise with stirring. 0.25 ml of 1 M pH9.5 carbonate buffer was added, and stirred for additional 3 hours, and then added with 0.25 ml of 0.2M lysine to mix well, and left at room temperature for 2 hours. Under stirring, an equal volume of saturated ammonium sulfate solution was added dropwise, and left at 4° C. for 1 hour. The resultants were centrifuged at 3000 rpm for half an hour, discarding the supernatant. The precipitate was washed with a half-saturated ammonium sulfate solution twice, and the final precipitate was dissolved in a small amount of 0.15 M pH 7.4 PBS. Above solution was then filled in a dialysis bag, and dialysed with 0.15 M pH 7.4 PBS buffer, and then centrifuged at 10000 rpm for 30 minutes to remove precipitate, wherein the supernatant is the enzyme conjugate, which was splitted and frozen stored.

The experiment was to observe the effect of different binding reaction times on the luminous quantity, using the invention and existing chemiluminescence detection techniques, respectively, with the binding reaction observed at time points of minute 1, 2, 4, 10, 20, 30, 45, and 60.

1. The Group of Existing Chemiluminescence Detection Technology

Corresponding to the 8 to-be-detected reaction time points, 8 EP tubes were taken, each added with 100 μl of magnetic particles labeled with an anti-human fibrinogen polyclonal antibody, and further added with 100 μl of a human fibrinogen solution of a concentration of 1 μg/ml, respectively. The binding reaction was allowed with shaking and incubating at 37° C. for 1 hour. The magnetic particles were adsorptively separated using a magnetic separator, discarding the supernatant, and washed by addition of 200 μl of PBS for three times. The magnetic particles were then adsorptively separated using a magnetic separator, discarding the supernatant, and added with 200 μl of a horseradish peroxidase labeled anti-human fibrinogen monoclonal antibody, allowing for a binding reaction at 37° C. with shaking and incubating for corresponding reaction times. The magnetic particles were adsorptively separated using a magnetic separator, discarding the supernatant, and washed by addition of 200 μl of PBS for three times, The magnetic particles were then adsorptively separated using a magnetic separator, discarding the supernatant, and transferred to a luminescence cup, which was then placed in a chemiluminescence detector, and added with 100 μl of the working solution of chemiluminescent substrate. When the reaction proceeded for 2 minutes, a luminous quantity of 6 seconds was recorded.

2. The Group of the Present Invention

The anti-human fibrinogen polyclonal antibody filter prepared in Example 1 was provided. 100 μl of a human fibrinogen solution with a concentration of 1 μg/ml was diluted to 1 ml with an alkaline PBS buffer (formulation: 50 mM disodium hydrogen phosphate, 50 mM potassium dihydrogen phosphate, and 150 mM sodium chloride, pH 8.0), and filtered, and the filter was washed with 1 ml of the alkaline PBS buffer by filtration for three times. Then, 100 μl of a horseradish peroxidase labeled anti-human fibrinogen monoclonal antibody was diluted to 1 ml with the alkaline PBS buffer, and filtered, and the filter was washed with 1 ml of the alkaline PBS buffer by filtration for three times. Then, it was filtered with 1 ml of pH 4.5 Tris-hydrochloric acid buffer (formulation: 50 mM Tris-HCl, 150 mM NaCl, pH4.5), and the filtrate was collected. 100 μl of the filtrate was pipetted to a luminescence cup, which was then placed in a chemiluminescence detector, and added with 100 μl of the working solution of chemiluminescent substrate. When a reaction proceeded for 2 minutes, a luminous quantity of 6 seconds was recorded, multiplied by 10, as a total counting result comparable to that of above existing chemiluminescence detection group. All of the filtrations involved in above process were controlled at a flow rate of 0.2 mL/min (the filtrations in all steps except for cleaning should be controlled at a flow rate of 0.05-1.0 mL/min).

The experiments were repeated three times, each experiment in triplicate, and the results were averaged.

III. Experimental Results

In the existing chemiluminescence detection, the luminous quantity (mV) was 3222, 5672, 7968, 9810, 14281, 20339, 19827, and 20513 at 1, 2, 4, 10, 20, 30, 45, and 60 minutes, respectively, and the binding reaction substantially reached equilibrium by 30 minutes, while the operation time of the whole process had been 89 minutes. The detection result of the present invention was 21320, with the operation time of the whole process being 13 minutes. Specific results of the three repeated experiments are shown in Table 1.

TABLE 1
Comparison results of luminous quantity as detection
performance between the present invention and the
existing chemiluminescence detection (unit: mV)

| | | Repeat 1 | Repeat 2 | Repeat 3 | Average |
|---|---|---|---|---|---|
| Existing | Minute 1 | 3257 | 2959 | 3450 | 3222 |
| chemilu- | Minute 2 | 5783 | 5541 | 5692 | 5672 |
| minescence | Minute 4 | 7735 | 8211 | 7958 | 7968 |
| detection | Minute 10 | 9296 | 10221 | 9913 | 9810 |
| technology | Minute 20 | 14653 | 14212 | 13978 | 14281 |
| | Minute 30 | 17581 | 21330 | 22106 | 20339 |
| | Minute 45 | 18026 | 21334 | 20121 | 19827 |
| | Minute 60 | 20719 | 21232 | 19588 | 20513 |

| | | | | |
|---|---|---|---|---|
| The present invention | 20494 | 22345 | 21121 | 21320 |

### Example 3

### Comparison of the Detection Results Between the Present Invention and the Existing Chemiluminescence Detection

I. Experimental Materials

The anti-human fibrinogen polyclonal antibody filter prepared in Example 1, the horseradish peroxidase labeled anti-human fibrinogen monoclonal antibody (prepared in Example 2), an anti-human fibrinogen polyclonal antibody (Commercially available from Yili Gaoke Bioengineering Institute, Beijing, China, Art. No. BR0812), magnetic particles (MP-COOH-20020, Yingnuo Biotechnology Co., Ltd., Zhengzhou, China), luminol, p-lodophenol, carbamide peroxide, a chemiluminescence detector (Promega, Glomax MultiJR Detection System), human fibrinogen (Commercially available from Sigma-Aldrich, under Produc Catalog No. F3879-1 G), plasma from healthy human (donated by a healthy volunteer).

II. Experimental Method

Formulation of a human fibrinogen solution: a known concentration of human fibrinogen solution was taken, and diluted with a PBS solution to formulate a series of human fibrinogen solutions of 10, 30, 70, 100, 300, 700 ng/ml, to make a standard curve.

Labeling of magnetic particles: in a conventional labeling way, magnetic particles were labeled with an anti-human fibrinogen polyclonal antibody, with a ratio (w/w) of the amounts of the anti-human fibrinogen polyclonal antibody and the magnetic particles of 1:10.

Formulation of a working solution of chemiluminescent substrate: a working solution of chemiluminescent substrate was formulated with luminol, p-lodophenol, carbamide peroxide, and the like. The working solution of chemiluminescent substrate had a solvent of water, and solutes and concentration thereof as follows: 0.36 mM luminol, 0.35 mM p-lodophenol, 4.5 mM carbamide peroxide. Each of above concentrations was a final concentration of the corresponding component in the solution.

In the experiments, the human fibrinogen solution was detected using the present invention and the existing chemiluminescence detection and standard curve was prepared, and then plasma was taken from healthy human, and 1000-fold diluted with PBS (50 mM disodium hydrogen phosphate, 50 mM potassium dihydrogen phosphate, 150 mM sodium chloride, pH 7.4), followed by measuring fibrinogen therein, and calculating the concentration of fibrinogen in the plasma from healthy human using the standard curve. 42 tubes were divided into a group of the present invention and a group of the existing chemiluminescence detection. Each sample was detected in triplicate.

1. The Group of the Existing Chemiluminescence Detection

Each of the tubes was charged with 100 μl of magnetic particles labeled with an anti-human fibrinogen polyclonal antibody, and added with 100 μl of a human fibrinogen solution or a dilution of plasma from healthy human, respectively, to allow for a binding reaction at 37° C. with shaking and incubating for 30 minutes, The magnetic particles were adsorptively separated using a magnetic separator, discarding the supernatant, and washed by addition of 200 μl of PBS for three times, The magnetic particles were then adsorptively separated using a magnetic separator, discarding the supernatant, and added with 200 μl of a horseradish peroxidase labeled anti-human fibrinogen monoclonal antibody, to allow for a binding reaction at 37° C. with shaking and incubating for 30 minutes. The magnetic particles were further adsorptively separated using a magnetic separator, discarding the supernatant, and washed by addition of 200 μl of PBS for three times. The magnetic particles were further adsorptively separated using a magnetic separator, discarding the supernatant, and transferred to a luminescence cup, which was then placed in a chemiluminescence detector, and added with 100 μl of the working solution of chemiluminescent substrate. When the reaction proceeded for 2 minutes, a luminous quantity of 6 seconds was recorded. A standard curve was plotted, and the content of human fibrinogen in the plasma was calculated.

2. The Group of the Present Invention

The anti-human fibrinogen polyclonal antibody filter prepared in Example 1 was provided. 100 μl of a human fibrinogen solution or a dilution of plasma from healthy human was diluted to 1 ml with an alkaline PBS buffer (formulation: 50 mM disodium hydrogen phosphate, 50 mM potassium dihydrogen phosphate, 150 mM sodium chloride, pH 8.0), and filtered, and the filter was washed with 1 ml of the alkaline PBS buffer by filtration for three times. Then, 100 μl of a horseradish peroxidase labeled anti-human fibrinogen monoclonal antibody was diluted to 1 ml with the alkaline PBS buffer, and filtered, and the filter was washed with 1 ml of the alkaline PBS buffer by filtration for three times. Then, it was filtered 1 ml of pH 4.5 Tris-hydrochloric acid buffer (formulation: 50 mM Tris-HCl, 150 mM NaCl, pH 4.5), and the filtrate was collected. 100 μl of the filtrate was pipetted to a luminescence cup, which was then placed in a chemiluminescence detector, and added with 100 μl of the working solution of chemiluminescent substrate. When the reaction proceeded for 2 minutes, a luminous quantity of 6 seconds was recorded. A standard curve was plotted, and the content of human fibrinogen in the plasma was calculated. All of the filtrations involved in above process were controlled at a flow rate of 0.3 mL/min (the filtrations in all steps except for cleaning should be controlled at a flow rate of 0.05-1.0 mL/min).

The experiments were repeated three times, and the results were averaged.

III. Experimental Results

The content of human fibrinogen in the plasma from healthy human was shown as 2.51 g/L in the existing chemiluminescence detection group, and as 2.58 g/L in the present invention group, that is, these two experimental methods showed the substantially identical results, with no statistical difference (P>0.05). However, the present invention (12 minutes) took significantly less time than that of the existing method (83 minutes) to finish the experiment. Specific results of the three repeated experiments are shown in Table 2.

TABLE 2
Comparison of the detection result between the present invention
and the existing chemiluminescence detection (unit: g/L)

| | Repeat 1 | Repeat 2 | Repeat 3 | Average |
|---|---|---|---|---|
| Existing chemilu- | 2.64 | 2.47 | 2.42 | 2.51 |
| minescence detection | | | | |
| The present invention | 2.7 | 2.55 | 2.49 | 2.58 |

### Example 4

### Effect of the Volume of the Filtration Core of the Present Invention on Detection Result

I. Experimental Materials

A self made long cylinder microfilter housing, NHS-activated agarose gel particles (CS-A30-01 NHS-activated Sepharose FF, Beijing Weishi Bohui Chromatography Technology Co., Ltd., China), an anti-human fibrinogen polyclonal antibody (commercially available from Yili Gaoke Bioengineering Institute, Beijing, China, Art. No. BR0812), sodium bicarbonate, hydrochloric acid, ethanolamine, human fibrinogen (Commercially available from Sigma-Aldrich, under Product Catalog No. F3879-1 G), a horseradish peroxidase labeled anti-human fibrinogen monoclonal antibody (prepared in Example 2).

II. Experimental Method

Formulation of a working solution of chemiluminescent substrate: a working solution of chemiluminescent substrate was formulated with luminol, p-lodophenol, carbamide peroxide, and the like. The working solution of chemiluminescent substrate had a solvent of water, and solutes and concentration thereof as follows: 0.36 mM luminol, 0.35 mm p-lodophenol, 4.5 mm carbamide peroxide. Each of above concentrations was a final concentration of the corresponding component in the solution.

Preparation of a series of anti-human fibrinogen polyclonal antibody filters: anti-human fibrinogen polyclonal antibody filters with a filtration core having a volume of 1, 2, 3, 5, 10, 50, 100, 300, 500, 700, 1000 mm³ respectively, as prepared by the method of Example 1, were used. In all of these filters, the filtration core is long-cylindrical, wherein a rigid plastic tube having an inner diameter of 1 mm was used in the 1, 2, 3, and 5 mm³ filters, a rigid plastic tube having an inner diameter of 2 mm was used on the 10 mm³ filter, a rigid plastic tube having an inner diameter of 5 mm was used in the 50, and 100 mm³ filters, and a rigid plastic tube having an inner diameter of 10 mm was used in the 300, 500, 700, and 1000 mm³ filters, respectively.

A human fibrinogen solution with a concentration of 1 μg/ml was formulated by dilution with a PBS solution (formulation: 50 mM disodium hydrogen phosphate, 50 mM potassium dihydrogen phosphate, 150 mM sodium chloride, pH7.4). 100 μl of the human fibrinogen solution was diluted with an alkaline PBS buffer (formulation: 50 mM disodium hydrogen phosphate, 50 mM potassium dihydrogen phosphate, 150 mM sodium chloride, pH 8.0) to 1 ml, i.e., a final concentration of 0.1 μg/ml.

The experiment was divided into two groups, a human fibrinogen group (Group A) and a control group (Group B). In the human fibrinogen group (Group A), 0.1 ml of the human fibrinogen solution with a final concentration of 0.1 μg/ml was filtered, and the filter was washed with 1 ml of the alkaline PBS buffer by filtration for three times. Then, 100 μl of a horseradish peroxidase labeled anti-human fibrinogen monoclonal antibody was diluted to 1 ml with the alkaline PBS buffer, and filtered, and the filter was washed with 1 ml of the alkaline PBS buffer by filtration for three times. Then, it was filtered with 1 ml of pH 4.5 Tris-hydrochloric acid buffer (formulation: 50 mM Tris-HCl, 150 mM NaCl, pH 4.5), and the filtrate was collected. 100 μl of a working solution of chemiluminescent substrate was taken for reaction, and when the reaction proceeded for 2 minutes, a luminous quantity of 6 seconds was recorded. The control group (Group B) is the same as the human fibrinogen group, except that a PBS buffer was used instead of 1 ml of the human fibrinogen solution with a final concentration of 0.1 μg/ml. The filtrations involved in above processes were manipulated at flow rates of 0.05 mL/min in the 1, 2, 3 and 5 mm³ filters, 0.1 mL/min in the 10 mm³ filter, 0.3 mL/min in the 50 and 100 mm³ filters, and 1.0 mL/min in the 300, 500, 700 and 1000 mm³ filters (the filtrations in all steps except for cleaning should be controlled at a flow rate of 0.05-1.0 mL/min).

The experiments were repeated three times, and the results were averaged.

III. Experimental Result

Recorded luminous quantities (mV) for the filters with a filtration core having volume of 1, 2, 3, 5, 10, 50, 100, 300, 500, 700, and 1000 mm³ were respectively 843, 1597, 1871, 1925, 1967, 1983, 2042, 1956, 1995, 2035, and 2068, wherein an equilibrium was substantially reached when the filtration core had a volume of 3 mm³. Specific results of the three repeats of the experiments are shown in Table 3.

TABLE 3
Effect of the volume of the filtration core of the present invention on
detection result - luminous quantity measurement (unit: mV)

| Filtration core | Repeat 1 | | Repeat 2 | | Repeat 3 | | Average |
|---|---|---|---|---|---|---|---|
| volume (mm³) | A | B | A | B | A | B | A-B |
| 1 | 1998 | 1022 | 2023 | 910 | 1522 | 1082 | 843 |
| 2 | 2809 | 1098 | 2566 | 1112 | 2692 | 1066 | 1597 |
| 3 | 3280 | 1101 | 2909 | 1046 | 2754 | 1183 | 1871 |
| 5 | 3122 | 1284 | 2990 | 1022 | 3167 | 1198 | 1925 |
| 10 | 3320 | 1398 | 3544 | 1536 | 3395 | 1424 | 1967 |
| 50 | 3122 | 1510 | 3968 | 1950 | 3855 | 1536 | 1983 |
| 100 | 3768 | 1860 | 3978 | 1862 | 3982 | 1880 | 2042 |
| 300 | 4533 | 2400 | 4255 | 2244 | 4326 | 2602 | 1956 |
| 500 | 5231 | 3160 | 5108 | 2698 | 4728 | 3224 | 1995 |
| 700 | 6854 | 5066 | 6723 | 4266 | 7126 | 5266 | 2035 |
| 1000 | 12530 | 11274 | 15217 | 11568 | 13135 | 11836 | 2068 |

### Example 5

### Comparison of Detection Result Between the Present Invention and Existing ELISA

I. Experimental Materials

An anti-human fibrinogen polyclonal antibody (Commercially available from Yili Gaoke Bioengineering Institute, Beijing, China, Art. No. BR0812), an anti-human fibrinogen polyclonal antibody filter prepared in Example 1, a horseradish peroxidase labeled anti-human fibrinogen monoclonal antibody (Prepared in Example 2), o-phenylenediamine, an ELISA detector (Bio-Rad, Model 550), human fibrinogen solution (Commercially available from Sigma-Aldrich, under Product Catalog No. F3879-1 G), plasma from healthy human (donated by a healthy volunteer).

II. Experimental Method

Formulation of a human fibrinogen solution: a known concentration of human fibrinogen solution was taken, and diluted with a PBS solution to formulate a series of human fibrinogen solutions of 30, 70, 100, 300, 700, 1000 ng/ml, to make a standard curve.

In the experiment, the present invention and the existing ELISA were used to detect the human fibrinogen solutions, and to plot a standard curve. Then, plasma is taken from healthy human, and 10000-fold diluted with PBS, followed by measuring fibrinogen therein, and calculating the concentration of fibrinogen in the plasma from healthy human using the standard curve. 42 tubes were divided into a group of the present invention and a group of the existing ELISA. Each sample was detected in triplicate.

1. The Group of Existing ELISA

A 96-well ELISA plate was used. Each of tubes was added 100 μl of an anti-human fibrinogen polyclonal antibody for coating at 4° C. overnight, after which each was washed for three times, and added with 100 μl of a human fibrinogen solution or a dilution of plasma from healthy human, respectively, to allow for a binding reaction at 37° C. with incubating for 120 minutes. Then, each was washed for three times, and added with 100 μl of a horseradish peroxidase labeled anti-human fibrinogen monoclonal antibody, to allow for a binding reaction at 37° C. with incubating for 60 minutes. Subsequently, each was washed for three times, the supernatant was discarded, and added with 100 μl of a color developing solution (formulation: 2.43 ml of 0.1 M citric acid, 2.57 ml of 0.2 M disodium hydrogen phosphate, 5 mg of o-phenylenediamine, 5 μl of hydrogen peroxide). After keeping in dark for 5 minutes, the resultants were added with 2M sulphuric acid to terminate the reaction. The resultants were placed on an ELISA detector to read absorbance value of OD 490, and the OD values corresponding to concentrations of 30, 70, 100, 300, 700, and 1000 ng/ml were, respectively, 0.198, 0.245, 0.256, 0.458, 0.895, and 1.256. A standard curve was plotted, and the content of human fibrinogen in the plasma was calculated.

2. The Group of the Present Invention

The anti-human fibrinogen polyclonal antibody filter prepared in Example 1 was provided. 100 μl of a human fibrinogen solution or a dilution of plasma from healthy human was each diluted to 1 ml with an alkaline PBS buffer, and filtered, and the filter was washed with 1 ml of the alkaline PBS buffer by filtration for three times. Then, 100 μl of a horseradish peroxidase labeled anti-human fibrinogen monoclonal antibody was diluted to 1 ml with the alkaline PBS buffer, and filtered, and the filter was washed with 1 ml of the alkaline PBS buffer by filtration for three times. Then, it was filtered with 1 ml of pH 4.5 Tris-hydrochloric acid, and the filtrate was collected. 100 μl of each of the filtrates was pipetted to a 96-well ELISA plate, and added with 100 μl of a color developing solution (formulation: 2.43 ml of 0.1 M citric acid, 2.57 ml of 0.2M disodium hydrogen phosphate, 5 mg of o-phenylenediamine, 5 μl of hydrogen peroxide). After keeping in dark for 5 minutes, the resultants were added with 2M sulphuric acid to terminate the reaction. The resultants were placed on an ELISA detector to read absorbance value of OD 490, and the OD values corresponding to concentrations of 30, 70, 100, 300, 700, and 1000 ng/ml were, respectively, 0.205, 0.255, 0.261, 0.455, 0.679, and 0.92. A standard curve was plotted, and the content of human fibrinogen in the plasma was calculated. All of the filtrations involved in above process were controlled at a flow rate of 0.1 mL/min (the filtrations in all steps except for cleaning should be controlled at a flow rate of 0.05-1.0 mL/min).

The experiments were repeated three times, and the results were averaged.

III. Experimental Result

The content of human fibrinogen in plasma from healthy human was shown as 2.56 g/L in the detection result of the existing ELISA, and as 2.50 g/L in the detection result of the present invention. That is, these two experimental methods showed the substantially identical results, with no statistical difference (P>0.05). However, the present invention (13 minutes) took significantly less time than that of the existing method (85 minutes) to finish the experiment. Specific results of the three repeats of the experiments are shown in Table 4.

TABLE 4
Comparison of the detection results between the present
invention and the existing ELISA (unit: g/L)

| | Repeat 1 | Repeat 2 | Repeat 3 | Average |
|---|---|---|---|---|
| Existing ELISA | 2.47 | 2.7 | 2.51 | 2.56 |
| The present invention | 2.44 | 2.61 | 2.45 | 2.50 |

### Example 6

### Analysis of the Detection Results of the Detection Method of the Present Invention Using Colloidal Gold as an Indicator

I. Experimental Materials

The anti-human fibrinogen polyclonal antibody filter prepared in Example 1, anti-human fibrinogen monoclonal antibody (Commercially available from Yili Gaoke Bioengineering Institute, Beijing, China, Art. No. BR0616), spectrophotometer (Jinghua Science and Technology Instrument Co., Ltd., Shanghai, China, 752 UV-Visible Spectrophotometer), human fibrinogen solution (Commercially available from Sigma-Aldrich, under Product Catalog No. F3879-1G), plasma from healthy human (donated by a healthy volunteer, the same as the plasma from healthy human in Example 5).

II. Experimental Method

Formulation of a human fibrinogen solution: a known concentration of human fibrinogen solution was taken, and diluted with a PBS solution to formulate a series of human fibrinogen solutions of 100, 300, 700, 1000, 3000 ng/ml, to make a standard curve.

Preparation of an colloidal gold labeled anti-human fibrinogen monoclonal antibody: 500 ml of pure water was taken and heated with stirring, and added with 500 μl of a solution of 10% chloroauric acid when water was boiled. After keeping boiled 5 minutes by heating, it was added with 500 μl of a 12% trisodium citrate solution, and kept the resulting solution boiled 10 minutes with stirring. Thereafter, the solution was naturally cooled to room temperature, to obtain a colloidal gold solution. A volume of 100 ml of the colloidal gold solution was adjusted to pH 8.3 with 10% potassium carbonate, and 1000 μg of an anti-human fibrinogen monoclonal antibody was quickly added thereto to a final concentration of 10 μg/ml. The beaker was shaken for mixing well, and left to stand at room temperature for 30 minutes, after which 1 ml of a 10% bovine serum albumin solution was quickly added, to form a final concentration of 1%, with simultaneously shaking the beaker, and then left at room temperature for 30 minutes, followed by centrifuging at 12000 rpm for 20 minutes. The supernatant was sucked out carefully, and the precipitate was added with 50 ml of a phosphate buffer to suspend, and centrifuged at 12000 rpm for 20 minutes. The supernatant was sucked out, and the precipitate was dissolved in 10.0 ml of a phosphate buffer containing 1% bovine serum albumin and 3% sucrose, and stored at 4° C. in dark.

In the experiment, the human fibrinogen solution was detected using the detect ion method of the present invention with colloidal gold as an indicator, and a standard curve was plotted. Then, plasma from healthy human was 5000-fold diluted with PBS, and measured, to thereby calculate the concentration of fibrinogen in the plasma from healthy human using the standard curve. 18 tubes of samples were used, with each sample detected in triplicate.

The anti-human fibrinogen polyclonal antibody filter prepared in Example 1 was provided. 100 μl of a human fibrinogen solution or a dilution of plasma from healthy human was each diluted to 1 ml with an alkaline PBS buffer, and filtered, and the filter was washed with 1 ml of the alkaline PBS buffer by filtration for three times. Then, 100 μl of a colloidal gold labeled anti-human fibrinogen monoclonal antibody was diluted with an alkaline PBS to 1 ml, and filtered, and the filter was washed with 1 ml of the alkaline PBS buffer by filtration for three times. Then, it was filtered with 1 ml of pH 4.5 Tris-hydrochloric acid, and the filtrate was collected. The filtrate was mixed well, and 800 μl of which was pipetted to spectrophotometer, to read the absorbance at a wavelength of 520 nm. A standard curve was plotted, and the content of human fibrinogen in the plasma was calculated. All of the filtrations involved in above process were controlled at a flow rate of 0.1 mL/min (the filtrations in all steps except for cleaning should be controlled at a flow rate of 0.05-1.0 mL/min).

The experiments were repeated three times, and the results were averaged.

III. Experimental Results

It was shown in the detection result of the detection method of the present invention with colloidal gold as an indicator that the content of human fibrinogen in the plasma from healthy human was 2.81 g/L, which was substantially identical with the result from other method (Example 5), with no statistical difference (P>0.05). Specific results of the three repeats of the experiments are shown in Table 5.

TABLE 5
Analysis of the detection results of the detection method of present
invention with colloidal gold as an indicator (unit: g/L)

| | Repeat 1 | Repeat 2 | Repeat 3 | Average |
|---|---|---|---|---|
| The present invention | 2.73 | 2.91 | 2.79 | 2.81 |
| (with colloidal | | | | |
| gold as an indicator) | | | | |

### Example 7

### Effect of the Shape of the Filter of the Present Invention on Detection Result

I. Experimental Materials

NHS-activated agarose gel particles (CS-A30-01 NHS-activated Sepharose FF, Beijing Weishi Bohui Chromatography Technology Co., Ltd., China), a horseradish peroxidase labeled anti-human fibrinogen monoclonal antibody (Prepared in Example 2), an anti-human fibrinogen polyclonal antibody (Commercially available from Yili Gaoke Bioengineering Institute, Beijing, China, Art. No. BR0812), sodium bicarbonate, hydrochloric acid, ethanolamine, human fibrinogen (Commercially available from Sigma-Aldrich, under Product Catalog No. F3879-1G), an ELISA detector (Bio-Rad, Model 550).

II. Experimental Method

A minitype long-cylindrical filter and a minitype short-cylindrical filter of an anti-human fibrinogen polyclonal antibody comprising a filtration core having a volume of 100 mm³ were prepared by the method of Example 1. The filtration core in the minitype long-cylindrical filter had a ratio of length to width (a ratio of height to bottom diameter of the cylinder) of 10:3, and the filtration core in the minitype short-cylindrical filter had a ratio of width to length (a ratio of bottom diameter to height of the cylinder) of 10:5.

A solution of 1 μg/ml human fibrinogen was formulated by dilution with a PBS solution. 100 μl of the human fibrinogen solution was diluted with an alkaline PBS buffer to 1 ml, and filtered, and the filter was washed with 1 ml of the alkaline PBS buffer by filtration for three times. Then, 100 μl of a horseradish peroxidase labeled anti-human fibrinogen monoclonal antibody was diluted to 1 ml with the alkaline PBS buffer, and filtered, and the filter was washed with 1 ml of the alkaline PBS buffer by filtration for three times. Then, it was further filtered with 1 ml of pH 4.5 Tris-hydrochloric acid, and the filtrate was collected. 100 μl of each of the filtrates was pipetted to a 96-well ELISA plate, added with 100 μl of an ELISA color developing solution (formulation: 2.43 ml of 0.1 M citric acid, 2.57 ml of 0.2M disodium hydrogen phosphate, 5 mg of o-phenylenediamine, 5 μl of hydrogen peroxide), and kept in dark for 3 minutes, and after which added with 2M sulphuric acid to terminate the reaction. The resultant was put on an ELISA detector to read its absorbance value at OD 490. All of the filtrations involved in above process were controlled at a flow rate of 1.0 mL/min (the filtrations in all steps except for cleaning should be controlled at a flow rate of 0.05-1.0 mL/min).

The experiments were repeated three times, and the results were averaged.

III. Experimental Results

It was determined that the absorbance value was 1.781 for the long-cylindrical filter, and 1.624 for the short-cylindrical filter. These two filters had the substantially same results, with no statistic difference (P>0.05). Specific results of the three repeats are shown in Table 6.

TABLE 6
Effect of the shape of the filter of the present
invention the Detection Result (value at OD 490)

| | Repeat 1 | Repeat 2 | Repeat 3 | Average |
|---|---|---|---|---|
| Long-cylindrical filter | 1.918 | 1.753 | 1.672 | 1.781 |
| Short-cylindrical filter | 1.494 | 1.765 | 1.613 | 1.624 |

### Example 8

### Comparison of Detection Results Between the Present Invention Using Alkaline Phosphatase Luminescent System and the Existing Chemiluminescence Detection

I. Experimental Materials

The anti-human fibrinogen polyclonal antibody filter prepared in Example 1, anti-human fibrinogen monoclonal antibody (Commercially available from Yili Gaoke Bioengineering Institute, Beijing, China, Art. No. BR0616), an anti-human fibrinogen polyclonal antibody (Commercially available from Yili Gaoke Bioengineering Institute, Beijing, China, Art. No. BR0812), alkaline phosphatase labeled donkey anti-mouse IgG (ComWin Biotech Co., Ltd., Beijing, China, CW0233), magnetic particles (MP-COOH-20020, Yingnuo Biotechnology Co., Ltd., Zhengzhou, China), APLS luminescent substrate solution (APLS0500, Yingnuo Biotechnology Co., Ltd., Zhengzhou, China), a chemiluminescence detector (Promega, Glomax MultiJR Detection System), human fibrinogen (Commercially available from Sigma-Aldrich, under Product Catalog No. F3879-1G), plasma from healthy human (donated by a healthy volunteer).

II. Experimental Method

Formulation of a human fibrinogen solution: a known concentration of human fibrinogen solution was taken, and diluted with a PBS solution to formulate 10, 30, 70, 100, 300, 700 ng/ml human fibrinogen solution, to make a standard curve.

Labeling of magnetic particles: in a conventional labeling way, magnetic particles were labeled with an anti-human fibrinogen polyclonal antibody, with a ratio (w/w) of the amounts of the anti-human fibrinogen polyclonal antibody and the magnetic particles of 1:10.

In the experiments, the human fibrinogen solution was detected using the present invention and the existing chemiluminescence detection technology and standard curve was plotted. Then, plasma from healthy human was 10000-fold diluted with PBS, to perform the measurement, and in turns to calculate the concentration of fibrinogen in the plasma from healthy human using the standard curve. 42 tubes were divided into a group of the present invention and a group of the existing chemiluminescence detection technology. Each sample was detected in triplicate.

1. The Group of the Existing Chemiluminescence detection

Each of the tubes was charged with 100 μl of magnetic particles labeled with an anti-human fibrinogen polyclonal antibody, and added with 100 μl of a human fibrinogen solution or a dilution of plasma from healthy human, respectively, to allow for a binding reaction at 37° C. with shaking and incubating for 30 minutes, The magnetic particles were adsorptively separated using a magnetic separator, discarding the supernatant, and washed by addition of 200 μl of PBS for three times. Then, the magnetic particles were adsorptively separated using a magnetic separator, discarding the supernatant, and added with 200 μl of a horseradish peroxidase labeled anti-human fibrinogen monoclonal antibody, to allow for a binding reaction at 37° C. with shaking and incubating for 30 minutes. Thereafter, the magnetic particles were adsorptively separated using a magnetic separator, discarding the supernatant, and washed by addition of 200 μl of PBS for three times. The magnetic particles were adsorptively separated using a magnetic separator, discarding the supernatant, and transferred to a luminescence cup, which was then placed in a chemiluminescence detector, and added with 100 μAPLS luminescent substrate solution. When the reaction proceeded 1 minute, a luminous quantity of 6 seconds was recorded. A standard curve was plotted, and the content of human fibrinogen in the plasma was calculated.

2. The Group of the Present Invention

The anti-human fibrinogen polyclonal antibody filter prepared in Example 1 was provided. 100 μl of a human fibrinogen solution or a dilution of plasma from healthy human was each diluted to 1 ml with an alkaline PBS buffer, and filtered, and the filter was washed with 1 ml of the alkaline PBS buffer by filtration for three times. Then, 100 μl of an anti-human fibrinogen monoclonal antibody was diluted with an alkaline PBS buffer to 1 ml, and filtered, and the filter was washed with 1 ml of the alkaline PBS buffer by filtration for three times. Then, 100 μl of alkaline phosphatase labeled donkey anti-mouse IgG was diluted with an alkaline PBS buffer to 1 ml, and filtered, and the filter was washed with 1 ml of the alkaline PBS buffer by filtration for three times. Then, it was further filtered with 1 ml of pH 4.5 Tris-hydrochloric acid, and the filtrate was collected. 100 μl of the filtrate was pipetted to a luminescence cup, which was then placed in a chemiluminescence detector, added with an APLS working solution of chemiluminescent substrate. When the reaction proceeded 1 minute, a luminous quantity of 6 seconds was recorded. A standard curve was plotted, and the content of human fibrinogen in the plasma was calculated. All of the filtrations involved in above process were controlled at a flow rate of 0.2 mL/min (the filtrations in all steps except for cleaning should be controlled at a flow rate of 0.05-1.0 mL/min).

The experiments were repeated three times, and the results were averaged.

III. Experimental Results

The content of human fibrinogen in the plasma from healthy human was shown as 2.32 g/L in the Detection Result of the existing chemiluminescence detection, and as 2.18 g/L in the detection result of the present invention using an alkaline phosphatase luminescent system. That is, these two experimental methods showed the substantially identical results, with no statistical difference (P>0.05). However, the present invention (13 minutes) took significantly less time than that of the existing method (86 minutes) to finish the experiment. Specific results of the three repeats of the experiments are shown in Table 7.

TABLE 7
Comparison of the detection results between the present invention
using an alkaline phosphatase luminescent system and the
existing chemiluminescence detection (unit: g/L)

| | Repeat 1 | Repeat 2 | Repeat 3 | Average |
|---|---|---|---|---|
| Existing chemilu- | 2.39 | 2.12 | 2.45 | 2.32 |
| minescence detection | | | | |
| Alkaline phosphatase | 2.1 | 2.33 | 2.11 | 2.18 |
| luminescent system | | | | |
| of the present invention | | | | |

### Example 9

### Comparison of Detection Results Between the Present Invention Directly Using the Filtration Core and the Existing Chemiluminescence Detection

I. Experimental Materials

The anti-human fibrinogen polyclonal antibody filter prepared in Example 1, anti-human fibrinogen monoclonal antibody (Commercially available from Yili Gaoke Bioengineering Institute, Beijing, China, Art. No. BR0616), an anti-human fibrinogen polyclonal antibody (Commercially available from Yili Gaoke Bioengineering Institute, Beijing, China, Art. No. BR0812), alkaline phosphatase labeled donkey anti-mouse IgG (ComWin Biotech Co., Ltd., Beijing, China, CW0233), magnetic particles (MP-COOH-20020, Yingnuo Biotechnology Co., Ltd., Zhengzhou, China), APLS luminescent substrate solution (APLS0500, Yingnuo Biotechnology Co., Ltd., Zhengzhou, China), a chemiluminescence detector (Promega, Glomax MultiJR Detection System), human fibrinogen (Commercially available from Sigma-Aldrich, under Produc Catalog No. F3879-1 G), plasma from healthy human (donated by a healthy volunteer).

II. Experimental Method

Formulation of a human fibrinogen solution: a known concentration of human fibrinogen solution was taken, and diluted with a PBS solution to formulate 10, 30, 70, 100, 300, 700 ng/ml human fibrinogen solution, to make a standard curve.

Labeling of magnetic particles: in a conventional labeling way, magnetic particles were labeled with an anti-human fibrinogen polyclonal antibody, with a ratio (w/w) of the amounts of the anti-human fibrinogen polyclonal antibody and the magnetic particles of 1:10.

In the experiments, the human fibrinogen solution was detected using the present invention and the existing chemiluminescence detection technology and a standard curve was plotted. Then, plasma from healthy human was 10000-fold diluted with PBS, to perform the measurement, and in turns to calculate the concentration of fibrinogen in the plasma from healthy human using the standard curve. 42 tubes were divided into a group of the present invention and a group of the existing chemiluminescence detection. Each sample was detected in triplicate.

1. The Group of the Existing Chemiluminescence Detection

Each of the tubes was charged with 100 μl of magnetic particles labeled with an anti-human fibrinogen polyclonal antibody, and added with 100 μl of a human fibrinogen solution or a dilution of plasma from healthy human, respectively, to allow for a binding reaction at 37° C. with shaking and incubating for 30 minutes. The magnetic particles were adsorptively separated using a magnetic separator, discarding the supernatant, and washed by addition of 200 μl of PBS for three times. The magnetic particles were adsorptively separated using a magnetic separator, discarding the supernatant, and added with 200 μl of a horseradish peroxidase labeled anti-human fibrinogen monoclonal antibody, to allow for a binding reaction at 37° C. with shaking and incubating for 30 minutes. The magnetic particles were adsorptively separated using a magnetic separator, discarding the supernatant, and washed by addition of 200 μl of PBS for three times. The magnetic particles were adsorptively separated using a magnetic separator, discarding the supernatant, and transferred to a luminescence cup, which was then placed in a chemiluminescence detector, and added with 100 μl of an APLS luminescent substrate solution. When the reaction proceeded 1 minute, a luminous quantity of 6 seconds was recorded. A standard curve was plotted, and the content of human fibrinogen in the plasma was calculated.

2. The Group of the Present Invention

The anti-human fibrinogen polyclonal antibody filter prepared in Example 1 was provided. 100 μl of a human fibrinogen solution or a dilution of plasma from healthy human was each diluted to 1 ml with an alkaline PBS buffer, and filtered, and the filter was washed with 1 ml of the alkaline PBS buffer by filtration for three times. Then, 100 μl of an anti-human fibrinogen monoclonal antibody was diluted with an alkaline PBS buffer to 1 ml, and filtered, and the filter was washed with 1 ml of the alkaline PBS buffer by filtration for three times. Then, 100 μl of alkaline phosphatase labeled donkey anti-mouse IgG was diluted with an alkaline PBS buffer to 1 ml, and filtered, and the filter was washed with 1 ml of the alkaline PBS buffer by filtration for three times. The filtration core was collected to a luminescence cup, which was then placed in a chemiluminescence detector, and added with an APLS working solution of chemiluminescent substrate. When the reaction proceeded 1 minute, a luminous quantity of 6 seconds was recorded. A standard curve was plotted, and the content of human fibrinogen in the plasma was calculated. All of the filtrations involved in above process were controlled at a flow rate of 0.1 mL/min (the filtrations in all steps except for cleaning should be controlled at a flow rate of 0.05-1.0 mL/min).

The experiments were repeated three times, and the results were averaged.

III. Experimental Result

The content of human fibrinogen in the plasma from healthy human was shown as 3.11 g/L in the detection result of the existing chemiluminescence detection, and as 3.28 g/L in the detection result of the present invention detection technology directly using the filtration core. That is, these two experimental methods showed the substantially identical results, with no statistical difference (P>0.05). However, the present invention took (13 minutes) significantly less time than that of the existing method (85 minutes) to finish the experiment. Specific results of the three repeats of the experiments are shown in Table 8.

TABLE 8
Comparison of the detection results between the present
invention directly using the filtration core and the
existing chemiluminescence detection (unit: g/L)

| | Repeat 1 | Repeat 2 | Repeat 3 | Average |
|---|---|---|---|---|
| Existing chemilu- | 2.96 | 3.12 | 3.25 | 3.11 |
| minescence detection | | | | |
| The present invention | 3.16 | 3.33 | 3.35 | 3.28 |
| directly using the | | | | |
| filtration core | | | | |

### Example 10

### Comparison of Detection Results Between the Present Invention Using an Effluent and the Existing Chemiluminescence Detection

I. Experimental Materials

The anti-human fibrinogen polyclonal antibody filter prepared in Example 1, anti-human fibrinogen monoclonal antibody (Commercially available from Yili Gaoke Bioengineering Institute, Beijing, China, Art. No. BR0616), an anti-human fibrinogen polyclonal antibody (Commercially available from Yili Gaoke Bioengineering Institute, Beijing, China, Art. No. BR0812), alkaline phosphatase labeled donkey anti-mouse IgG (ComWin Biotech Co., Ltd., Beijing, China, CW0233), magnetic particles (MP-COOH-20020, Yingnuo Biotechnology Co., Ltd., Zhengzhou, China), APLS luminescent substrate solution (APLS0500, Yingnuo Biotechnology Co., Ltd., Zhengzhou, China), a chemiluminescence detector (Promega, Glomax MultiJR Detection System), human fibrinogen solution (Commercially available from Sigma-Aldrich, under Produc Catalog No. F3879-1G), plasma from healthy human (donated by a healthy volunteer).

II. Experimental Method

Formulation of a human fibrinogen solution: a known concentration of human fibrinogen solution was taken, and diluted with a PBS solution to formulate 10, 30, 70, 100, 300, 700 ng/ml human fibrinogen solution, to make a standard curve.

Labeling of magnetic particles: in a conventional labeling way, magnetic particles were labeled with an anti-human fibrinogen polyclonal antibody, with a ratio (w/w) of the amounts of the anti-human fibrinogen polyclonal antibody and the magnetic particles of 1:10.

In the experiments, the human fibrinogen solution was detected using the present invention and the existing chemiluminescence detection technology. Then, plasma from healthy human was 10000-fold diluted with PBS, to perform the measurement, and in turns to calculate the concentration of fibrinogen in the plasma from healthy human using the standard curve. 42 tubes were divided into a group of the present invention and a group of the existing chemiluminescence detection. Each sample was detected in triplicate.

1. The Group of the Existing Chemiluminescence Detection

Each of the tubes was charged with 100 μl of magnetic particles labeled with an anti-human fibrinogen polyclonal antibody, and added with 100 μl of a human fibrinogen solution or a dilution of plasma from healthy human, respectively, to allow for a binding reaction at 37° C. with shaking and incubating for 30 minutes, The magnetic particles were adsorptively separated using a magnetic separator, discarding the supernatant, and washed by addition of 200 μl of PBS for three times. The magnetic particles were adsorptively separated using a magnetic separator, discarding the supernatant, and added with 200 μl of a horseradish peroxidase labeled anti-human fibrinogen monoclonal antibody, to allow for a binding reaction at 37° C. with shaking and incubating for 30 minutes. The magnetic particles were adsorptively separated using a magnetic separator, discarding the supernatant, and washed by addition of 200 μl of PBS for three times. The magnetic particles were adsorptively separated using a magnetic separator, discarding the supernatant, and transferred to a luminescence cup, which was then placed in a chemiluminescence detector, and added with 100 μl of an APLS luminescent substrate solution. When the reaction proceeded 1 minute, a luminous quantity of 6 seconds was recorded. A standard curve was plotted, and the content of human fibrinogen in the plasma was calculated.

2. The Group of the Present Invention

The anti-human fibrinogen polyclonal antibody filter prepared in Example 1 was provided. 100 μl of a human fibrinogen solution or a dilution of plasma from healthy human was each diluted to 1 ml with an alkaline PBS buffer, and filtered, and the filter was washed with 1 ml of the alkaline PBS buffer by filtration for three times. Then, 100 μl of an anti-human fibrinogen monoclonal antibody was diluted with an alkaline PBS buffer to 1 ml, and filtered, and the filter was washed with 1 ml of the alkaline PBS buffer by filtration for three times. Then, 20 μl of alkaline phosphatase labeled donkey anti-mouse IgG was diluted with an alkaline PBS buffer to 1 ml, and filtered, and the filtrate was collected to luminescence cup, which was then placed in a chemiluminescence detector, added with an APLS working solution of chemiluminescent substrate. When the reaction proceeded 1 minute, a luminous quantity of 6 seconds was recorded. A standard curve was plotted, and the content of human fibrinogen in the plasma was calculated. All of the filtrations involved in above process were controlled at a flow rate of 0.5 mL/min (the filtrations in all steps except for cleaning should be controlled at a flow rate of 0.05-1.0 mL/min).

The experiments were repeated three times, and the results were averaged.

III. Experimental Results

The content of human fibrinogen in the plasma from healthy human was shown as 3.02 g/L in the detection result of the existing chemiluminescence detection, and as 3.31 g/L in the detection result of the present invention using an effluent. That is, these two experimental methods showed the substantially identical results, with no statistical difference (P>0.05). However, the present invention (12 minutes) took significantly less time than that of the existing method (88 minutes) to finish the experiment. Specific results of the three repeats of the experiments are shown in Table 9.

TABLE 9
Comparison of the Detection Results between the
Present Invention using an Affluent and the Existing
Chemiluminescence Detection (unit: g/L)

| | Repeat 1 | Repeat 2 | Repeat 3 | Average |
|---|---|---|---|---|
| Existing chemilu- | 2.79 | 3.01 | 3.26 | 3.02 |
| minescence detection | | | | |
| The present invention | 3.34 | 3.48 | 3.11 | 3.31 |
| using an effluent | | | | |

### Example 11

### Comparison of Detection Results Between the Present Invention and the Existing Chemiluminescence Detection Technology

I. Experimental Materials

As compared with Example 3, “the anti-human fibrinogen polyclonal antibody filter prepared in Example 1” was replaced with “the filtration detection device prepared in Example 1”, wherein the detector was a chemiluminescence detector, and remaining experimental materials were the same as in Example 3.

II. Experimental Method

1. The Group of the Existing Chemiluminescence Detection

Same as Example 3.

2. The Group of the Present Invention

The filtration detection device prepared in Example 1 was provided. 100 μl of a human fibrinogen solution or a dilution of plasma from healthy human was diluted to 1 ml with an alkaline PBS buffer (formulation: 50 mM disodium hydrogen phosphate, 50 mM potassium dihydrogen phosphate, 150 mM sodium chloride, pH 8.0), as a solution of a sample to be detected, and placed in a reservoir for sample to be detected. The solution of the sample to be detected was flowed into a filter via a silicone tube through a peristaltic pump, for filtration. Next, 1 ml of an alkaline PBS buffer was added to the reservoir for sample to be detected, to wash the filter three times by filtration. Then, 100 μl of a horseradish peroxidase labeled anti-human fibrinogen monoclonal antibody was diluted with an alkaline PBS buffer to 1 ml, as a solution of a detection material, and placed in a detection phase reservoir. The solution of the detection material was flowed into the filter via a silicone tube through a peristaltic pump, for filtration. Next, 1 ml of an alkaline PBS buffer was added to the detection phase reservoir, to wash the filter three times by filtration. Finally, 1 ml of a pH 4.5 Tris-hydrochloric acid buffer (formulation: 50 mM Tris-HCl, 150 mM NaCl, pH 4.5) was added in the detection phase reservoir, and filtered, and the filtrate was collected. 100 μl of the filtrate was pipetted to a luminescence cup, which was then placed in a chemiluminescence detector, and added with 100 μl of the working solution of chemiluminescent substrate. When the reaction proceeded for 2 minutes, a luminous quantity of 6 seconds was recorded. A standard curve was plotted, and the content of human fibrinogen in the plasma was calculated. All of the filtrations involved in above process were controlled at a flow rate of 0.3 mL/min (the filtrations in all steps except for cleaning should be controlled at a flow rate of 0.05-1.0 mL/min).

The experiments were repeated three times, and the results were averaged.

III. Experimental Results

The content of human fibrinogen in the plasma from healthy human was shown as 2.51 g/L in the existing chemiluminescence detection group, and as 2.70 g/L in the detection result of the present invention. That is, these two experimental methods showed the substantially identical results, with no statistical difference (P>0.05), However, the present invention (12 minutes) took significantly less time than that of the existing method (87 minutes) to finish the experiment. Specific results of the three repeats of the experiments are shown in Table 10.

TABLE 10
Comparison of the detection results between the present invention
and the existing chemiluminescence detection technology (unit: g/L)

| | Repeat 1 | Repeat 2 | Repeat 3 | Average |
|---|---|---|---|---|
| Existing chemilu- | 2.44 | 2.43 | 2.66 | 2.51 |
| minescence detection | | | | |
| The present invention | 3.04 | 2.65 | 2.41 | 2.70 |

### INDUSTRIAL APPLICATION

The present invention designs a detection device which uses a filter as a reaction carrier in a creative way and improves detection customization controllability and detection efficiency. The filtration of the present invention has a reaction time significantly shorter than that of the existing detections, and thus improves detection speed. The present invention can complete the whole reaction at room temperature without a temperature-controlled reaction structure required in an existing detection device, thus simplifying instrument design, and realizing miniaturization and portability. Therefore, the technique of the present invention has a good application prospect and great significance in improving existing immunoassay techniques.

What is claimed:

1. A filtration detection device, characterized by: comprising a reservoir for sample to be detected, a detection phase reservoir, a filter and a detector; the filter consisting of an inlet, a filtration layer and an outlet, the filtration layer having a filtration core that is a solid phase material coupling with a specific binder to an object to be detected; the reservoir for sample to be detected and the detection phase reservoir respectively communicating with the inlet of the filter via a pipe.

2. The filtration detection device according to claim 1, characterized by that: the detector is an instrument that enables the quantitative detection and recordation of the color or light quantity variation.

3. The filtration detection device according to claim 1 or 2, characterized by that:
the solid phase material serving as the filtration core is a particulate matter or a mesh-like material.

4. The filtration detection device according to any one of claims 1-3, characterized by that: the filtration core has a volume of 2 mm³ to 1 cm³.

5. The filtration detection device according to claim 4, characterized by that: the filtration core has a volume of 3 mm³ to 0.3 cm³.

6. The filtration detection device according to claim 5, characterized by that: the filtration core has a volume of 5 mm³ to 0.1 cm³.

7. The filtration detection device according to any one of claims 1-6, characterized by that: the filtration core is shaped having length greater than width, with a ratio of length to width of 2-100.

8. The filtration detection device according to claim 7, characterized by that: the filtration core is shaped having length greater than width, with a ratio of length to width of 2-50.

9. The filtration detection device according to claim 8, characterized by that: the filtration core is shaped having length greater than width, with a ratio of length to width of 2-30.

10. The filtration detection device according to any one of claims 1-6, characterized by that: the filtration core is shaped having width greater than length, with a ratio of width to length of 1.1-10.

11. The filtration detection device according to claim 10, characterized by that: the filtration core is shaped having width greater than length, with a ratio of width to length of 1.1-5.

12. The filtration detection device according to claim 10, characterized by that: the filtration core is shaped having width greater than length, with a ratio of width to length of 1.1-3.

13. The filtration detection device according to any one of claims 1-12, characterized by that: each pipe has a pump structure fixed thereon.

14. A method for determining the content of an object to be detected in a sample to be detected using the filtration detection device according to any one of claims 1-13, as following (A) or (B):
(A) comprising steps of:
1) placing solution A into the reservoir for sample to be detected; placing solution B into the detection phase reservoir; the solution A being a solution of a sample to be detected containing the object to be detected; the solution B being a solution of detection material, the detection material being a material formed from a specific binder of an object to be detected, labeled with an indicator; the indicator being a substance capable of directly or indirectly resulting in a color or light quantity variation;
2) flowing the solution A into the filter through a pipe, so that the object to be detected is specifically captured by the filtration core;
3) flowing the solution B into the filter through a pipe, so that the detection material is specifically bound with the object to be detected that is specifically captured by the filtration core, to form complex 1 of the filtration core, the object to be detected, and the detection material;
4) detecting, as any of following (a)-(c):
(a) detecting the filtration core with the captured object to be detected and detection material directly using a detector, to calculate the content of the object to be detected through a color or light quantity variation produced by the indicator;
(b) eluting the filter with an eluent, collecting the eluent containing the detection material, detecting the eluent with the detector for a color or light quantity variation of the indicator, to thereby calculate the content of the object to be detected;
(c) detecting the solution B flowing out of the filter directly using a detector for a color or light quantity variation of the indicator, to thereby calculate the content of the object to be detected;
(B) comprising steps of:
1) placing solution A into the reservoir for sample to be detected; placing solution B1 into the detection phase reservoir; the solution A being a solution of a sample to be detected containing the object to be detected; the solution B1 being a solution of a specific binder of the object to be detected, not labeled with an indicator;
2) flowing the solution A into the filter through a pipe, so that the object to be detected is specifically captured by the filtration core;
3) flowing the solution B1 into the filter through a pipe, so that the unlabeled specific binder of the object to be detected is specifically bound with the object to be detected specifically captured by the filtration core, to form complex 2 of the filtration core, the object to be detected, and the unlabeled specific binder of the object to be detected;
4) placing solution B2 into the detection phase reservoir; the solution B2 being a solution of a material labeled with an indicator, the material labeled with the indicator being capable of binding with the specific binder of the object to be detected in the solution B1; the indicator being a substance capable of directly or indirectly resulting in a color or light quantity variation;
5) flowing the solution B2 into the filter through a pipe, so that the material labeled with the indicator is bound with the unlabeled specific binder of the object to be detected in the complex 2, to form complex 3 of the filtration core, the object to be detected, the unlabeled specific binder of the object to be detected, and the material labeled with the indicator;
6) detecting, as any of following (a)-(c):
(a) detecting the filtration core with the captured object to be detected, the unlabeled specific binder of the object to be detected and the material labeled with the indicator directly using a detector, to calculate the content of the object to be detected through a color or light quantity variation produced by the indicator;
(b) eluting the filter with an eluent, collecting the eluent containing the material labeled with the indicator, detecting the eluent with the detector for a color or light quantity variation of the indicator, to thereby calculate the content of the object to be detected;
(c) detecting the solution B2 flowing out of the filter directly using a detector for a color or light quantity variation of the indicator, to thereby calculate the content of the object to be detected.

15. The method according claim 14, characterized by that: the method further comprising a step of cleaning the filter after step 2) and/or step 3).

16. A filter, characterized by: consisting of an inlet, a filtration layer and an outlet, the filtration layer having a filtration core that is a solid phase material coupling with a specific binder of an object to be detected.

17. The filter according to claim 16, characterized by that: the solid phase material serving as the filtration core is a particulate matter or a mesh-like material.

18. The filter according to claim 16 or 17, characterized by that: the filtration core has a volume of 2 mm³ to 1 cm³.

19. The filter according to claim 18, characterized by that: the filtration core has a volume of 3 mm³ to 0.3 cm³.

20. The filter according to claim 19, characterized by that: the filtration core has a volume of 5 mm³ to 0.1 cm³.

21. The filter according to any one of claims 16-20, characterized by that: the filtration core is shaped having length greater than width, with a ratio of length to width of 2-100.

22. The filter according to claim 21, characterized by that: the filtration core is shaped having length greater than width, with a ratio of length to width of 2-50.

23. The filter according to claim 22, characterized by that: the filtration core is shaped having length greater than width, with a ratio of length to width of 2-30.

24. The filter according to any one of claims 16-20, characterized by that: the filtration core is shaped having width greater than length, with a ratio of width to length of 1.1-10.

25. The filter according to claim 24, characterized by that: the filtration core is shaped having width greater than length, with a ratio of width to length of 1.1-5.

26. The filter according to claim 25, characterized by that: the filtration core is shaped having width greater than length, with a ratio of width to length of 1.1-3.

27. A method for determining the content of an object to be detected in a sample to be detected using the filter according to any one of claims 16-26, as following (C) or (D):
(C) comprising steps of:
1) injecting solution A into the filter, the solution A being a solution of a sample to be detected containing the object to be detected, so that the object to be detected is specifically captured by the filtration core;
2) injecting solution B into the filter, the solution B being a solution of the detection material, the detection material being a material formed from a specific binder of an object to be detected, labeled with an indicator; the indicator being a substance capable of directly or indirectly resulting in a color or light quantity variation, so that the detection material is specifically bound with the object to be detected that is specifically captured by the filtration core, to form complex 1 of the filtration core, the object to be detected, and the detection material;
3) detecting, as any of following (a)-(c):
(a) detecting the filtration core with the captured object to be detected and detection material directly using a detector, to calculate the content of the object to be detected through a color or light quantity variation produced by the indicator;
(b) eluting the filter with an eluent, collecting the eluent containing the detection material, and detecting a color or light quantity variation of the indicator in the eluent using a detector, to thereby calculate the content of the object to be detected;
(c) detecting the detection material solution flowing out of the filter directly using a detector for a color or light quantity variation of the indicator, to thereby calculate the content of the object to be detected;
(D) comprising steps of:
1) injecting solution A into the filter, the solution A being a solution of a sample to be detected containing the object to be detected, so that the object to be detected is specifically captured by the filtration core;
2) injecting solution B1 to the filter, the solution B1 being a solution of a specific binder of the object to be detected, not labeled with an indicator, so that the unlabeled specific binder of the object to be detected is specifically bound with the object to be detected specifically captured by the filtration core, to form complex 2 of the filtration core, the object to be detected, and the unlabeled specific binder of the object to be detected;
3) injecting solution B2 into the filter, the solution B2 being a solution of a material labeled with an indicator, that is capable of binding to the unlabeled specific binder of the object to be detected in the solution B1; the indicator being a substance capable of directly or indirectly resulting in a color or light quantity variation, so that the material labeled with the indicator is bound with the unlabeled specific binder of the object to be detected in the complex 2, to form complex 3 of the filtration core, the object to be detected, the unlabeled specific binder of the object to be detected, and the material labeled with the indicator;
6) detecting, as any of following (a)-(c):
(a) detecting the filtration core with the captured object to be detected, the unlabeled specific binder of the object to be detected and the material labeled with the indicator directly using a detector, to calculate the content of the object to be detected through a color or light quantity variation produced by the indicator;
(b) eluting the filter with an eluent, collecting the eluent containing the material labeled with the indicator, and detecting a color or light quantity variation of the indicator in the eluent using a detector, to thereby calculate the content of the object to be detected;
(c) detecting the solution B2 flowing out of the filter directly using a detector for a color or light quantity variation of the indicator, to thereby calculate the content of the object to be detected.

28. The method according to claim 27, characterized by: further comprising a step of cleaning the filter after step 1) and/or step 2).

29. Use of the filtration detection device according to any one of claims 1-13 or the filter according to any one of claims 16-26 in the manufacture of a product for a quantitative immunoassay.
